(19) United States
(12) Patent Application Publication
(10) Pub. No.: US 2008/0248130 A1
(43) Pub. Date: Oct. 9, 2008

(54) Composition and Method For the Promotion of Hair Growth on a mammal

(76) Inventors: Matthias Rath, Haarlem (NL); Aleksandm Niedzwiecki, San Jose, CA (US); Waheed Roomi, Sunnyvale, CA (US)

Correspondence Address: INHOUSE CO., 280 COLORADO AVE., PALO ALTO, CA, 94301, US

(21) Appl. No.: 11/696,725

(22) Filed: Apr. 5, 2007

(51) Int. Cl. A61K 33/34; A61K 31/401

(52) U.S. Cl. 424/638; 514/423

(57) ABSTRACT

Administration of composition to a mammal comprising lysine, proline, arginine, an ascorbic compound, and polyphenols in an effective amount promotes hair growth of the mammal. Administration the composition for promotion of hair growth in a mammal further comprises calcium, magnesium, N-acetyl-cysteine, selenium, copper, and manganese. Use of a composition comprising lysine, proline, arginine, an ascorbic compound, and polyphenols in an effective amount may be used for the preparation of a compound for promoting hair growth in a mammal.

### FIELD OF THE INVENTION

The present invention is generally related to a method of promoting hair growth in a mammal, and more particularly related to a novel use of composition comprising ascorbic compound, lysine, proline, and polyphenols for promoting hair growth.

### BACKGROUND OF THE INVENTION

It was disclosed in one U.S. Pat. No. 6,331,569 that a variety of amino acids such as proline and lysine for oral administration may improve hair growth. According to disclosure of another U.S. Pat. No. 6,908,941, alkanolamine was used in connection with an ascorbic acid for enhancement of hair and nail growth. However, none of the prior art disclosure teaches or suggests the use of a composition comprising lysine, proline, arginine, an ascorbic compound, and polyphenols to promote hair growth.

Despite extensive research and development focusing on hair growth agents, hair loss prevention and hair growth effects and the like, the conventional hair growth agents has not always been effective. A number of hair growth stimuli were developed from growth factors, hormones, plant extracts, and a combination thereof with limited success achieved so far.

Accordingly, there is always a need to find alternative method of promoting hair growth which slightly differ in biological properties, specificity or other characteristic. First, this is due to the fact that not all promising methods result in an effective promotion of hair growth, for example due to low bioavailability or unwanted side-effects. Secondly, there is a need for alternative therapies if a patient does not respond to a first method. The present application provides alternative method for the promotion of hair growth.

Currently available treatments for promoting hair growth include minoxidil (Rogaine®), which is available without a prescription. It is applied to the scalp. Both men and women can use it. However, an increase in the absorption of minoxidil from the scalp can occur in patients with inflamed or abnormal scalps, leading to side effects of a fall in blood pressure, an increase in the heart rate, and weight gain.

Another medicine, finasteride (Propecia®) is available with a prescription. It comes in pills and is only for men and may have a side effect of impairing male sexual function. Both medicines may take up to 6 months before you can tell if one of these medicines is working.

In effect, there is a long-felt and on going need for a method of promoting hair growth in humans using composition comprising naturally occurring ingredients free of the side effects mentioned above.

The present invention satisfies that need.

### SUMMARY OF THE INVENTION

The present invention is based, at least in part, on the discovery that a composition comprising lysine, proline, arginine, an ascorbic compound, and polyphenols will promote hair growth in a mammal.

Accordingly, in one aspect, the invention provides methods of promoting hair growth of a mammal by administering a composition to the mammal comprising lysine, proline, arginine, an ascorbic compound, and polyphenols in an effective amount.

In one embodiment, the composition is comprised of lysine, proline, arginine, ascorbic acid, calcium, magnesium, polyphenols, N-acetyl-cysteine, selenium, copper, and manganese. In another embodiment, the composition is comprised of approximately 400-1500 mg lysine, 500-1500 mg proline, 200-1000 mg arginine, 400-1500 mg of ascorbic compound, 5-50 mg calcium, 10-100 mg magnesium, 500-2000 mg polyphenols, 100-500 mg N-acetyl-cysteine, 5-60 μg selenium, 0.5-5 mg copper, and 0.5-3 mg manganese. In a preferred embodiment, the composition is comprised of approximately 1000 mg lysine, 750 mg proline, 500 mg arginine, 710 mg of ascorbic compound, 22 mg calcium, 50 mg magnesium, 800 mg polyphenols, 200 mg N-acetyl-cysteine, 30 μg selenium, 2 mg copper, and 1 mg manganese.

In another aspect, the invention provides a use of a composition comprising lysine, proline, arginine, an ascorbic compound, and polyphenols in an effective amount for the preparation of a compound for promoting hair growth in a mammal.

### BRIEF DESCRIPTION OF THE DRAWINGS

FIG. 1A is a picture of the shaved back of a mouse from Group I on day zero.

FIG. 1B is a picture of the back of a mouse from Group I after feeding the mouse with regular diet and olive oil applied three times a week on the back of the mouse for 3 weeks.

FIG. 1C is a picture of the back of a mouse from Group I after feeding the mouse with regular diet and olive oil applied three times a week on the back of the mouse for 4 weeks.

FIG. 2A is a picture of the shaved back of a mouse from Group II on day zero.

FIG. 2B is a picture of the back of a mouse from Group II after feeding the mouse with regular diet and 250 mg of composition of Example 1 applied three times a week on the back of the mouse for 3 weeks.

FIG. 2C is a picture of the back of a mouse from Group II after feeding the mouse with regular diet and 250 mg of composition of Example 1 applied three times a week on the back of the mouse for 4 weeks.

FIG. 3A is a picture of the shaved back of a mouse from Group III on day zero.

FIG. 3B is a picture of the back of a mouse from Group III after feeding the mouse with regular diet supplemented with 0.5% of composition of Example 1 for 3 weeks.

FIG. 3C is a picture of the back of a mouse from Group III after feeding the mouse with regular diet supplemented with 0.5% of composition of Example 1 for 4 weeks.

### DETAILED DESCRIPTION OF THE INVENTION

As used herein, the article “a” or “an” means one or more than one (that is, at least one) of the grammatical object of the article, unless otherwise made clear in the specific use of the article in only a singular sense.

As used herein, “mammal” means any of various warm-blooded vertebrate animals of the class mammalia, including humans, characterized by a covering of hair on the skin and, in the female, milk-producing mammary glands for nourishing the young.

An amount is “effective” as used herein, when the amount provides an effect in promoting hair growth in the subject. For those skilled in the art, the effective amount, as well as dosage and frequency of administration, may easily be determined according to their knowledge and standard methodology of merely routine experimentation based on the present disclosure.

The practice of the present invention may employ, unless otherwise indicated, conventional techniques in various fields. Specific illustrations of suitable techniques can be had by reference to the examples herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed. W.H. Freeman Pub., New York, N.Y. and Berg et al. (2002) Biochemistry, 5^{th} Ed., W.H. Freeman Pub., New York, N.Y., Short Protocols in Molecular Biology, 3rd Ed., ed. by Ausubel, F. et al. Wiley, NY (1995), Methods In Enzymology Academic Press, Inc., N.Y., Immunochemical Methods In Cell And Molecular Biology Mayer and Walker, eds., Academic Press, London (1987), Handbook Of Experimental Immunology, Volumes I-IV D. M. Weir and C. C. Blackwell, eds. (1986), all of which are herein incorporated in their entirety by reference for all purposes.

### EXAMPLES

### Example 1

This example illustrates the preparation of a representative composition containing the biochemical compounds listed in the following table (Table 1).

TABLE 1

| Biochemical Substances | Units | Amount |
|---|---|---|
| L-Lysine | mg | 1000 |
| L-Proline | mg | 750 |
| L-Arginine | mg | 500 |
| Ascorbic Acid | mg | 710 |
| Calcium | mg | 22 |
| Magnesium | mg | 50 |
| Polyphenols | mg | 1000 |
| N-acetyl-cysteine | mg | 200 |
| Selenium | μg | 30 |
| Copper | mg | 2 |
| Manganese | mg | 1 |

mg = milligram
μg = microgram

### Example 2

To demonstrate the utility of the composition of the invention to promote growth in a mammal, we evaluate the composition of these biochemical compounds for its ability to induce hair growth of mice.

A total of 15 Male Swiss Webster mice 4-5 weeks of age on arrival were purchased from Simonsen Laboratory, Gilroy, Calif. US. They were housed in cages under pathogen-free conditions on 12-hour light/12-hour dark schedule for a week. The mice were then divided into three groups I, II and III, five mice per group.

Before starting the experiment, the hair on the back of the mice were (clipped and carefully shaved. Initial pictures were taken and the mice were then kept on different regiment as follows.

Group I—Control group was fed the regular diet, and olive oil (0.5 ml) was applied three times a week on the shaved area of the mice for four weeks.

Group II—Test group was fed the regular diet. Composition of 250 mg of Example 1 of current invention in 0.5 ml olive oil was applied on the shaved area of the mice three times a week for four weeks.

Group III—Test group was fed the regular diet supplemented with 0.5% of composition of Example 1 for four weeks.

Some mice were sacrificed at the end of three weeks and the rest at four weeks. Skin was excised and processed for histology. Pictures were taken at three and four week, before the mice were sacrificed.

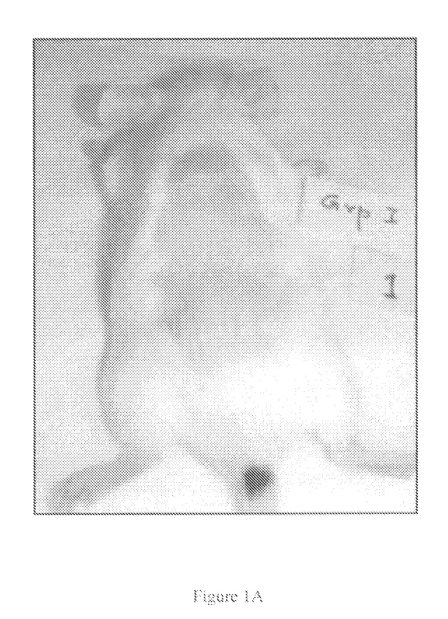

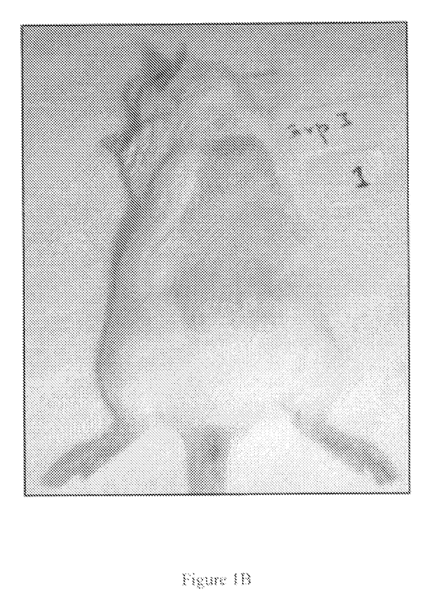

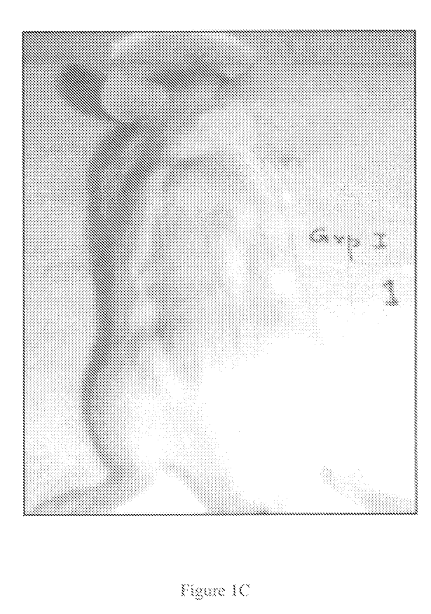

FIG. 1A is a picture of the shaved back of a mouse from Group I on day zero. FIG. 1B is a picture of the back of a mouse from Group I after feeding the mouse with regular diet and olive oil applied three times a week on the back of the mouse for 3 weeks. FIG. 1C is a picture of the back of a mouse from Group I after feeding the mouse with regular diet and olive oil applied three times a week on the back of the mouse for 4 weeks.

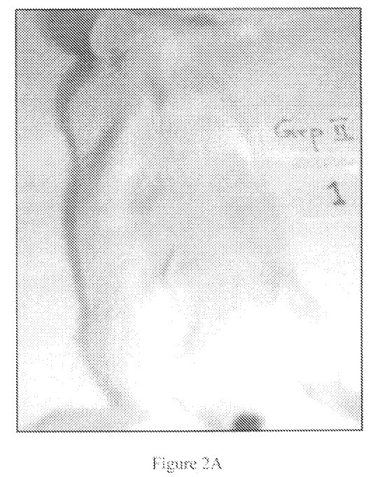

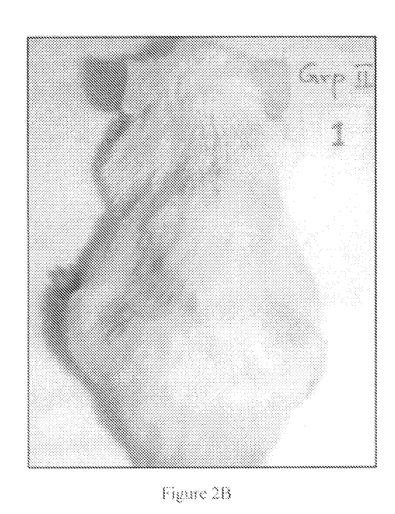

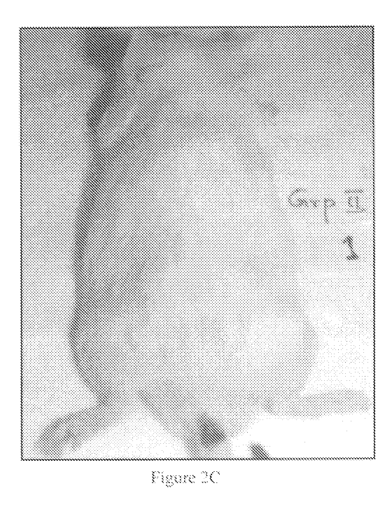

FIG. 2A is a picture of the shaved back of a mouse from Group II on day zero. FIG. 2B is a picture of the back of a mouse from Group II after feeding the mouse with regular diet and 250 mg of composition of Example 1 applied three times a week on the back of the mouse for 3 weeks. FIG. 2C is a picture of the back of a mouse from Group II after feeding the mouse with regular diet and 250 mg of composition of Example 1 applied three times a week on the back of the mouse for 4 weeks.

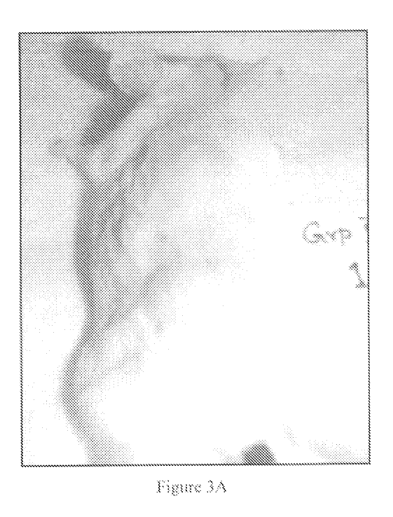

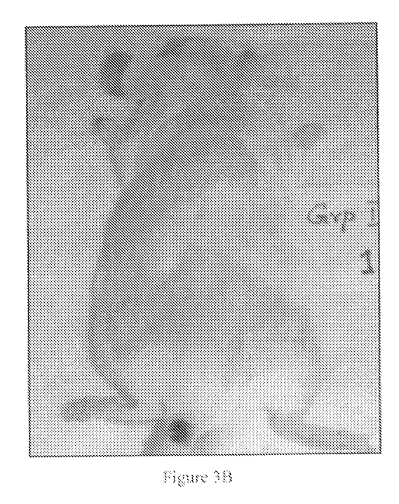

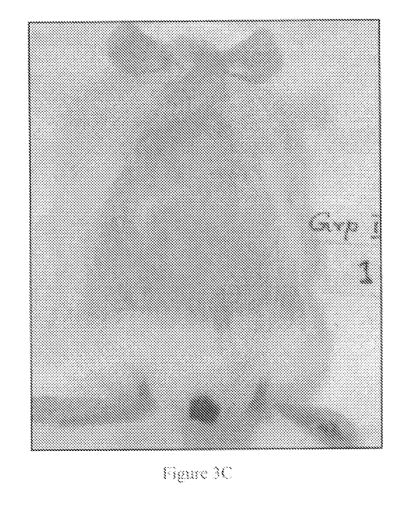

FIG. 3A is a picture of the shaved back of a mouse from Group III on day zero. FIG. 3B is a picture of the back of a mouse from Group III after feeding the mouse with regular diet supplemented with 0.5% of composition of Example 1 for 3 weeks. FIG. 3C is a picture of the back of a mouse from Group III after feeding the mouse with regular diet supplemented with 0.5% of composition of Example 1 for 4 weeks.

Comparing FIG. 1A, FIG. 2A and FIG. 3A, it is clear that similar amount of hair was removed from the different groups of mice.

Results of the study, as illustrated in FIG. 1C, FIG. 2C and FIG. 3C demonstrate that administration of composition in Example 1 affected mice's ability the grow hair. Specifically, the composition in Example 1 promotes hair growth as demonstrated by comparing FIG. 1C, FIG. 2C and FIG. 3C.

In the experiment, control group was not exposed to the composition listed in Example 1. After 4 weeks, only marginal amount of hair grew back as demonstrated by FIG. 1C. Group II (FIG. 2C) after 4 weeks experienced the best result as the hair on the back of the mouse was completely grown back. Group III (FIG. 3C) after 4 weeks also experienced additional hair growth as compared to the control group.

As such, the composition listed in Table 1 is surprising in its effectiveness in promoting hair growth in a mammal. The effect of the composition listed in Table 1 on hair growth in the different group of mice demonstrates a significant therapeutic use of the composition. Accordingly, based on the experimental results, one of skill in the art would recognize that application of a composition in accordance with the invention will be effective in promoting hair growth in a mammal. The above examples clearly show that composition of Table 1 is effective in promoting hair growth of a mammal by administering such composition to the mammal.

While the present invention has been described with reference to specific embodiments thereof, it should be understood by those skilled in the art that various changes may be made and equivalents may be substituted without departing from the true spirit and scope of the invention. One of ordinary skill in the art would appreciate that the effective amounts of the biochemical compounds may vary depending on the variations in patients, durations of treatment etc. Modifications may be made to adapt a particular situation, and composition of matter. A number of embodiments of the invention have been described in the present application; nevertheless, it will be understood all such modifications are intended to be within the scope of the following claims.

The contents of all references, pending patent applications and published patents, cited throughout this application are hereby expressly incorporated by reference. It is to be understood that the above description is intended to be illustrative and not restrictive. Those skilled in the art will recognize, or be able to ascertain using no more than routine experimentation, many equivalents to the specific embodiments of the invention described herein. Such equivalents are intended to be encompassed by the following claims.

What is claimed is:

1. A method for promoting hair growth in a mammal by administering to the mammal a composition comprising lysine, proline, arginine, an ascorbic compound, and polyphenols in an effective amount.

2. The method according to claim 1, wherein the composition further comprises calcium, magnesium, N-acetyl-cysteine, selenium, copper, and manganese.

3. The method according to claim 2, wherein the composition comprising about 400 to about 1500 mg lysine, about 500 to about 1500 mg proline, about 200 to about 1000 mg arginine, about 400 to about 1500 mg of ascorbic compound, about 5 to about 50 mg calcium, about 10 to about 100 mg magnesium, about 500 to about 2000 mg polyphenols, about 100 to about 500 my N-acetyl-cysteine, about 5 to about 60 μg selenium, about 0.5 to about 5 mg copper, and about 0.5 to about 3 mg manganese.

4. The method according to claim 2, wherein the composition comprising about 1000 mg lysine, about 750 m proline, about 500 mg arginine, about 710 mg of ascorbic compound, about 22 mg calcium, about 50 mg magnesium, about 800 mg polyphenols, about 200 mg N-acetyl-cysteine, about 30 μg selenium, about 2 mg copper, and about 1 mg manganese.

5. Use of a composition comprising lysine, proline, arginine, an ascorbic compound, and polyphenols in an effective amount for the preparation of a compound for promoting hair growth in a mammal.

6. The method according to claim 5, wherein the composition further comprises calcium, magnesium, N-acetyl-cysteine, selenium, copper, and manganese.

7. The method according to claim 6, wherein the composition comprising about 400 to about 1500 mg lysine, about 500 to about 1500 mg proline, about 200 to about 1000 mg arginine, about 400 to about 1500 mg of ascorbic compound, about 5 to about 50 mg calcium, about 10 to about 100 mg magnesium, about 500 to about 2000 mg polyphenols, about 100 to about 500 mg N-acetyl-cysteine, about 5 to about 60 μg selenium, about 0.5 to about 5 mg coppers and about 0.5 to about 3 mg manganese.

8. The method according to claim 6, wherein the composition comprising about 1000 mg lysine, about 750 m proline, about 500 mg arginine, about 710 mg of ascorbic compound, about 22 mg calcium, about 50 mg magnesium, about 800 mg polyphenols, about 200 mg N-acetyl-cysteine, about 30 μg selenium, about 2 mg copper, and about 1 my manganese.
